(19) United States
(12) Patent Application Publication
(10) Pub. No.: US 2025/0258371 A1
(43) Pub. Date: Aug. 14, 2025

(54) Gaze Tracking Circuitry with Optical Range Finders

(71) Applicant: Apple Inc., Cupertino, CA (US)

(72) Inventor: Patrick R Gill, Victoria (CA)

(21) Appl. No.: 18/984,695

(22) Filed: Dec. 17, 2024

Related U.S. Application Data
(60) Provisional application No. 63/551,876, filed on Feb. 9, 2024.

(51) Int. Cl. G02B 27/00; G01S 7/48; G01S 17/48; G01S 17/86; G02B 27/01

(52) U.S. Cl. CPC G02B 27/0093; G01S 7/4808; G01S 17/48; G01S 17/86; G02B 27/0172; G02B 2027/0174; G02B 2027/0178

(57) ABSTRACT

Eyewear such as a head-mounted device may include adjustable prescription lenses and/or may include displays. The eyewear may include gaze tracking circuitry that tracks a gaze direction of a user. The gaze tracking circuitry may include a range finder that uses phase-based optical coherence tomography to determine eye distance. The range finder may include one or more emitters such as lasers that emit infrared light into a beam splitter that splits the light into signal light that travels a free space path to the eye and reference light that travels a fixed reference path towards multiple image sensors located around the periphery of the eye. The signal light specularly reflects off of the eye (creating an eye glint) and the reflected signal light combines with the reference light, creating an interference pattern that can be detected by the image sensors and analyzed to determine eye distance.

This application claims the benefit of provisional patent application No. 63/551,876, Feb. 9, 2024, which is hereby incorporated by reference herein in its entirety.

### FIELD

This relates generally to electronic devices, and, more particularly, to wearable electronic devices such as head-mounted devices.

### BACKGROUND

Head-mounted devices and other eyewear may use gaze tracking circuitry to track a user's gaze.

It can be challenging to design gaze tracking circuitry that performs satisfactorily. If care is not taken, the gaze tracking circuitry may produce inaccurate measurements or may exhibit other performance limitations such as excessive power consumption.

### SUMMARY

Eyewear such as a head-mounted device may include adjustable prescription lenses and/or may include displays. The lenses and displays may be mounted to a support structure such as supporting frames or other head-mounted support structures.

The eyewear may include gaze tracking circuitry that tracks a gaze direction of a user. The gaze tracking circuitry may include a camera, a light source, and a range finder, or the gaze tracking circuitry may include only a range finder.

The range finder may use phase-based optical coherence tomography to determine eye distance. The range finder may include one or more emitters that each include one or more lasers or other coherent light sources. The lasers may emit infrared light of known, time-dependent wavelength into a beam splitter that splits the light into signal light that travels a free space path length to the eye and reference light that travels a fixed reference path length towards multiple image sensors. The signal light specularly reflects off of the eye (creating an eye glint) and the reflected signal light combines with the reference light, creating an interference pattern that can be captured by the image sensor and analyzed to determine glint location, eye distance, eye velocity, and eye acceleration.

### BRIEF DESCRIPTION OF THE DRAWINGS

FIG. 1 is a top view of an illustrative head-mounted device that may include gaze tracking circuitry in accordance with an embodiment.

FIG. 2 is a rear view of an illustrative head-mounted device that may include gaze tracking circuitry in accordance with an embodiment.

FIG. 3 is a schematic diagram of an illustrative head-mounted device that may include gaze tracking circuitry in accordance with an embodiment.

FIG. 4 is a top view of illustrative gaze tracking circuitry being used to track a gaze direction of a user in accordance with an embodiment.

FIG. 5 is a schematic diagram of an illustrative range finder that uses optical coherent tomography to determine eye distance in accordance with an embodiment.

FIG. 6 is a rear view of an illustrative head-mounted device having a range finder of the type shown in FIG. 5 in accordance with an embodiment.

FIG. 7 is a side view of an illustrative range finder having an image sensor that includes a pixel array, a holographic optical element, a phase grating, and a lens in accordance with an embodiment.

FIG. 8 is a side view of an illustrative phase grating having edges that are offset from the vertex of an overlapping lens in accordance with an embodiment.

FIG. 9 is a front view of an illustrative image captured by a pixel array and having two focal spots that are slightly out of phase with one another in accordance with an embodiment.

### DETAILED DESCRIPTION

Eyewear such as a pair of glasses or other head-mounted device may include one or more eye monitoring components such as gaze tracking circuitry for determining the direction of a user's gaze. The gaze tracking circuitry may include one or more range finders (e.g., distance sensors) that use optical coherence tomography to measure the path lengths of specular reflections off of the sclera and cornea of the eye (sometimes referred to as glints). This path length information may in turn be used to determine the direction of a user's gaze. The gaze tracking circuitry may rely entirely on distance sensing to track the direction of the user's gaze, or the gaze tracking circuitry may use both distance sensors and one or more cameras for capturing images of the pupil and one or more glints in a hybrid graze tracking approach.

A distance sensor based on optical coherence tomography may include one or more coherent light sources such as lasers that emit beams of infrared light into a beam splitter. The beams of infrared light may have a wavelength that changes slightly over time in a known or measured way. The beam splitter may include a star coupler that allows light from each laser to be detected by multiple sensors in different locations around the periphery of the eye. The beam splitter may split the infrared light into signal light that travels a free space path to the eye and reference light that travels a fixed reference path to one or more image sensors. The signal light that is emitted toward the eye creates a specular reflection (e.g., a glint) on the eye and is reflected toward the image sensor. The reflected signal light from the eye combines with the reference light from the fixed reference path and a corresponding interference pattern is captured by the image sensor. If the free space path length to the eye is equal to the fixed reference path length, the image of the glint will appear unchanged. If the two path lengths are different, a corresponding interference pattern may be captured by the image sensor and analyzed to determine glint location, eye distance, as well as eye velocity and acceleration (e.g., the first two derivatives of eye distance).

In a hybrid gaze tracking arrangement, a range finder based on optical coherence tomography may be combined with a light source and camera for determining pupil location and glint location. For example, a light source such as a light-emitting diode may create a glint on the user's eye and/or may illuminate the user's pupil and iris. Pupil location and glint location may be determined based on the eye images captured by the camera. The range finder may use phase-based optical coherence tomography to determine a distance to the eye (sometimes referred to as eye distance). The gaze direction of the user may be determined based on the location of the glint, the location of the pupil, and the distance to the eye.

Using hybrid gaze tracking circuitry that includes both a camera and light source for glint detection and a range finder for distance sensing may allow gaze direction to be determined using fewer glints (e.g., using only a single glint from a single light-emitting diode, if desired). This may be beneficial in arrangements where fewer light sources are desired and/or in scenarios where multiple glints cannot be obtained from a particular user's eye. In some arrangements, the camera, light source, and range finder may remain powered on during operation of device 10 and may continuously or periodically be used to track gaze direction. In other arrangements, power savings may be achieved by keeping the camera and/or the light source off (or otherwise in a low-power state) until the range finder detects a change in eye distance, which may be indicative of a change in gaze direction. Upon detecting the change in eye distance with the range finder, the camera and light source may be switched on to help determine the new gaze direction of the user.

In some arrangements, glint detection may not be necessary to determine gaze direction. For example, a camera can be used to capture information about the pupil, iris, eyelids, and/or other features of the eye, and this information can be supplemented with one or more range measurements from an optical coherence tomographic measurement of a path length of a specular reflection off of the surface of the user's eye.

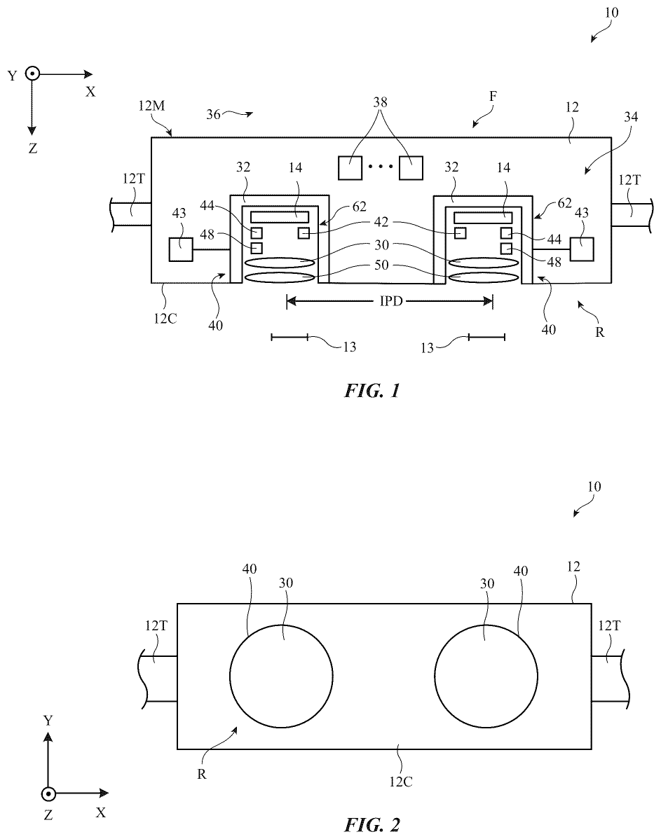

A top view of an illustrative head-mounted device or other eyewear is shown in FIG. 1. As shown in FIG. 1, head-mounted devices such as electronic device 10 may have head-mounted support structures such as housing 12. Housing 12 may include portions (e.g., support structures 12T) to allow device 10 to be worn on a user's head. Support structures 12T may be formed from fabric, polymer, metal, and/or other material. Support structures 12T may form a strap or other head-mounted support structures to help support device 10 on a user's head. A main support structure (e.g., main housing portion 12M) of housing 12 may support electronic components such as displays 14. Main housing portion 12M may include housing structures formed from metal, polymer, glass, ceramic, and/or other material. For example, housing portion 12M may have housing walls on front face F and housing walls on adjacent top, bottom, left, and right side faces that are formed from rigid polymer or other rigid support structures and these rigid walls may optionally be covered with electrical components, fabric, leather, or other soft materials, etc. The walls of housing portion 12M may enclose internal components 38 in interior region 34 of device 10 and may separate interior region 34 from the environment surrounding device 10 (exterior region 36). Internal components 38 may include integrated circuits, actuators, batteries, sensors, and/or other circuits and structures for device 10. Housing 12 may be configured to be worn on a head of a user and may form glasses, a hat, a helmet, goggles, and/or other head-mounted device. Configurations in which housing 12 forms goggles may sometimes be described herein as an example.

Front face F of housing 12 may face outwardly away from a user's head and face. Opposing rear face R of housing 12 may face the user. Portions of housing 12 (e.g., portions of main housing 12M) on rear face R may form a cover such as cover 12C (sometimes referred to as a curtain). The presence of cover 12C on rear face R may help hide internal housing structures, internal components 38, and other structures in interior region 34 from view by a user.

Device 10 may have left and right optical modules 40. Each optical module may include a respective display 14, lens 30, and support structure 32. Support structures 32, which may sometimes be referred to as lens barrels or optical module support structures, may include hollow cylindrical structures with open ends or other supporting structures to house displays 14 and lenses 30. Support structures 32 may, for example, include a left lens barrel that supports a left display 14 and left lens 30 and a right lens barrel that supports a right display 14 and right lens 30.

Displays 14 may include arrays of pixels or other display devices to produce images. Displays 14 may, for example, include organic light-emitting diode pixels formed on substrates with thin-film circuitry and/or formed on semiconductor substrates, pixels formed from crystalline semiconductor dies, liquid crystal display pixels, scanning display devices, and/or other display devices for producing images.

Lenses 30 may include one or more lens elements for providing image light from displays 14 to respective eyes boxes 13. Lenses 30 may be implemented using refractive glass lens elements, using mirror lens structures (catadioptric lenses), using Fresnel lenses, using holographic lenses, and/or other lens systems.

When a user's eyes are located in eye boxes 13, displays (display panels) 14 operate together to form a display for device 10 (e.g., the images provided by respective left and right optical modules 40 may be viewed by the user's eyes in eye boxes 13 so that a stereoscopic image is created for the user). The left image from the left optical module fuses with the right image from a right optical module while the display is viewed by the user.

If desired, device 10 may include additional lenses such as lenses 50. Lenses 50 may be fixed lenses or may be adjustable lenses such as liquid crystal lenses, fluid-filled lenses, or other suitable adjustable lenses. Lenses 50 may be configured to accommodate different focal ranges and/or to correct for vision defects such as myopia, hyperopia, presbyopia, astigmatism, higher-order aberrations, and/or other vision defects. For example, lenses 50 may be adjustable prescription lenses having a first set of optical characteristics for a first user with a first prescription and a second set of optical characteristics for a second user with a second prescription. Lenses 50 may be removably or permanently attached to housing 12. In arrangements where lenses 50 are removable, lenses 50 may have mating engagement features, magnets, clips, or other attachment structures that allow lenses 50 to be attached to housing 12 (e.g., individually or as a pair).

If desired, device 10 may be used purely for vision correction (e.g., device 10 may be a pair of spectacles, glasses, etc.) and some of the other components in FIG. 1 such as displays 14, lenses 30, and optical modules 40 may be omitted. In other arrangements, device 10 (sometimes referred to as eyewear 10, glasses 10, head-mounted device 10, etc.) may include displays that display virtual reality, mixed reality, and/or augmented reality content. With this type of arrangement, lenses 50 may be prescription lenses and/or may be used to move content between focal planes from the perspective of the user. If desired, lenses 50 may be omitted. Arrangements in which device 10 is a head-mounted device with one or more displays are sometimes described herein as an illustrative example.

It may be desirable to monitor the user's eyes while the user's eyes are located in eye boxes 13. For example, it may be desirable to use a camera to capture images of the user's irises (or other portions of the user's eyes) for user authentication. It may also be desirable to monitor the direction of the user's gaze. Gaze tracking information may be used as a form of user input and/or may be used to determine where, within an image, image content resolution should be locally enhanced in a foveated imaging system. To ensure that device 10 can capture satisfactory eye images while a user's eyes are located in eye boxes 13, each optical module 40 may be provided with gaze tracking circuitry 62. Gaze tracking circuitry 62 may include one or more cameras such as camera 42, one or more light sources such as light source 44 (e.g., light-emitting diodes, lasers, lamps, etc.), and one or more range finders such as range finder 48. Device 10 may include gaze tracking circuitry 62 for each eye (e.g., a left eye and a right eye), or device 10 may include gaze tracking circuitry 62 for a single eye.

Cameras 42 and light-emitting diodes 44 may operate at any suitable wavelengths (visible, infrared, and/or ultraviolet). With an illustrative configuration, which may sometimes be described herein as an example, diodes 44 emit infrared light that is invisible (or nearly invisible) to the user. This allows eye monitoring operations to be performed continuously without interfering with the user's ability to view images on displays 14.

Range finder 48 (sometimes referred to as depth sensor 48) may be any suitable range finder such as an optical range finder (e.g., a light source and light sensor that gather time-of-flight measurements, phase-based measurements, self-mixing sensors, light detection and ranging (lidar) sensors, structured light sensors, and/or depth sensors based on stereo imaging devices that capture three-dimensional images, etc.), an ultrasonic range finder (e.g., one or more capacitive micromachined ultrasonic transducers, piezoelectric micromachined transducers, and/or other suitable ultrasonic transducers for emitting and/or detecting acoustic signals), and/or any other suitable range finder.

In some arrangements, range finder 48 may use phase-based optical coherence tomography to determine a distance to the eye. With this type of arrangement, range finder 48 may include one or more coherent light sources such as wavelength-tunable lasers that emit light into a beam splitter while sweeping a wavelength of the emitted light across a given range of tunability. Laser light that is emitted with this type of wavelength sweep may sometimes be referred to as a laser chirp. The beam splitter splits the light into a signal arm that creates a specular reflection (e.g., a glint) on the user's eye and a local oscillator arm that will interfere with the light collected from the signal arm. Range finder 48 may include one or more image sensors (e.g., arrays of pixels) that capture an image of the signal light and reference light, which may include an interference pattern based on the constructive and destructive interference between the signal light from the eye and the reference light from the local oscillator arm. The captured images may be processed to determine the path length of the specular reflection and the corresponding distance to the eye. If desired, only a portion of the pixel array that includes the glint image may be read out for processing (e.g., to save processing power). In particular, a first laser chirp may be used to identify the portion of the pixel array that includes the glint, and a second laser chirp may be used to determine a distance to the glint using only that portion of the pixel array (e.g., by only reading out a subset of the pixels that includes the glint). If desired, a third laser chirp may be used to determine the first temporal derivative of the distance to the eye (e.g., eye velocity). If desired, third and fourth laser chirps may be used to determine the first two temporal derivatives of the distance to the eye (e.g., eye velocity and eye acceleration).

Not all users have the same interpupillary distance IPD. To provide device 10 with the ability to adjust the interpupillary spacing between modules 40 along lateral dimension X and thereby adjust the spacing IPD between eye boxes 13 to accommodate different user interpupillary distances, device 10 may be provided with actuators 43. Actuators 43 can be manually controlled and/or computer-controlled actuators (e.g., computer-controlled motors) for moving support structures 32 relative to each other. Information on the locations of the user's eyes may be gathered using, for example, cameras 42. The locations of eye boxes 13 can then be adjusted accordingly.

As shown in FIG. 2, cover 12C may cover rear face F while leaving lenses 30 of optical modules 40 uncovered (e.g., cover 12C may have openings that are aligned with and receive modules 40). As modules 40 are moved relative to each other along dimension X to accommodate different interpupillary distances for different users, modules 40 move relative to fixed housing structures such as the walls of main portion 12M and move relative to each other.

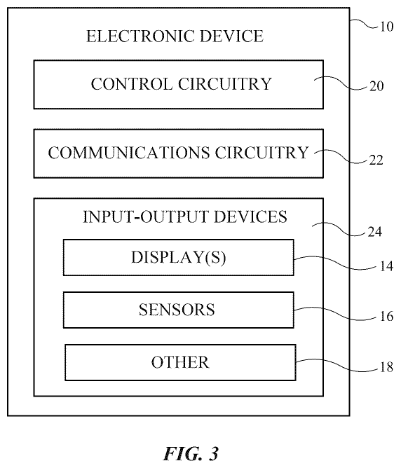

A schematic diagram of an illustrative electronic device such as a head-mounted device or other wearable device is shown in FIG. 3. Device 10 of FIG. 3 may be operated as a stand-alone device and/or the resources of device 10 may be used to communicate with external electronic equipment. As an example, communications circuitry in device 10 may be used to transmit user input information, sensor information, and/or other information to external electronic devices (e.g., wirelessly or via wired connections). Each of these external devices may include components of the type shown by device 10 of FIG. 3.

As shown in FIG. 3, a head-mounted device such as device 10 may include control circuitry 20. Control circuitry 20 may include storage and processing circuitry for supporting the operation of device 10. The storage and processing circuitry may include storage such as nonvolatile memory (e.g., flash memory or other electrically-programmable-read-only memory configured to form a solid state drive), volatile memory (e.g., static or dynamic random-access-memory), etc. Processing circuitry in control circuitry 20 may be used to gather input from sensors and other input devices and may be used to control output devices. The processing circuitry may be based on one or more microprocessors, microcontrollers, digital signal processors, baseband processors and other wireless communications circuits, power management units, audio chips, application specific integrated circuits, etc. During operation, control circuitry 20 may use display(s) 14 and other output devices in providing a user with visual output and other output.

To support communications between device 10 and external equipment, control circuitry 20 may communicate using communications circuitry 22. Circuitry 22 may include antennas, radio-frequency transceiver circuitry, and other wireless communications circuitry and/or wired communications circuitry. Circuitry 22, which may sometimes be referred to as control circuitry and/or control and communications circuitry, may support bidirectional wireless communications between device 10 and external equipment (e.g., a companion device such as a computer, cellular telephone, or other electronic device, an accessory such as a point device, computer stylus, or other input device, speakers or other output devices, etc.) over a wireless link. For example, circuitry 22 may include radio-frequency transceiver circuitry such as wireless local area network transceiver circuitry configured to support communications over a wireless local area network link, near-field communications transceiver circuitry configured to support communications over a near-field communications link, cellular telephone transceiver circuitry configured to support communications over a cellular telephone link, or transceiver circuitry configured to support communications over any other suitable wired or wireless communications link. Wireless communications may, for example, be supported over a Bluetooth® link, a WiFi® link, a wireless link operating at a frequency between 10 GHz and 400 GHz, a 60 GHz link, or other millimeter wave link, a cellular telephone link, or other wireless communications link. Device 10 may, if desired, include power circuits for transmitting and/or receiving wired and/or wireless power and may include batteries or other energy storage devices. For example, device 10 may include a coil and rectifier to receive wireless power that is provided to circuitry in device 10.

Device 10 may include input-output devices such as devices 24. Input-output devices 24 may be used in gathering user input, in gathering information on the environment surrounding the user, and/or in providing a user with output. Devices 24 may include one or more displays such as display(s) 14. Display(s) 14 may include one or more display devices such as organic light-emitting diode display panels (panels with organic light-emitting diode pixels formed on polymer substrates or silicon substrates that contain pixel control circuitry), liquid crystal display panels, microelectromechanical systems displays (e.g., two-dimensional mirror arrays or scanning mirror display devices), display panels having pixel arrays formed from crystalline semiconductor light-emitting diode dies (sometimes referred to as microLEDs), and/or other display devices.

Sensors 16 in input-output devices 24 may include force sensors (e.g., strain gauges, capacitive force sensors, resistive force sensors, etc.), audio sensors such as microphones, touch and/or proximity sensors such as capacitive sensors such as a touch sensor that forms a button, trackpad, or other input device), and other sensors. If desired, sensors 16 may include optical sensors such as optical sensors that emit and detect light, ultrasonic sensors, optical touch sensors, optical proximity sensors, and/or other touch sensors and/or proximity sensors, monochromatic and color ambient light sensors, image sensors, fingerprint sensors, iris scanning sensors, retinal scanning sensors, and other biometric sensors, temperature sensors, sensors for measuring three-dimensional non-contact gestures (“air gestures”), pressure sensors, sensors for detecting position, orientation, and/or motion (e.g., accelerometers, magnetic sensors such as compass sensors, gyroscopes, and/or inertial measurement units that contain some or all of these sensors), health sensors such as blood oxygen sensors, heart rate sensors, blood flow sensors, and/or other health sensors, radio-frequency sensors, depth sensors (e.g., structured light sensors and/or depth sensors based on stereo imaging devices that capture three-dimensional images), optical sensors such as self-mixing sensors and light detection and ranging (lidar) sensors that gather time-of-flight measurements, humidity sensors, moisture sensors, gaze tracking sensors, electromyography sensors to sense muscle activation, facial sensors, and/or other sensors. In some arrangements, device 10 may use sensors 16 and/or other input-output devices to gather user input. For example, buttons may be used to gather button press input, touch sensors overlapping displays can be used for gathering user touch screen input, touch pads may be used in gathering touch input, microphones may be used for gathering audio input, accelerometers may be used in monitoring when a finger contacts an input surface and may therefore be used to gather finger press input, etc.

If desired, electronic device 10 may include additional components (see, e.g., other devices 18 in input-output devices 24). The additional components may include haptic output devices, actuators for moving movable housing structures, audio output devices such as speakers, light-emitting diodes for status indicators, light sources such as light-emitting diodes that illuminate portions of a housing and/or display structure, other optical output devices, and/or other circuitry for gathering input and/or providing output. Device 10 may also include a battery or other energy storage device, connector ports for supporting wired communication with ancillary equipment and for receiving wired power, and other circuitry.

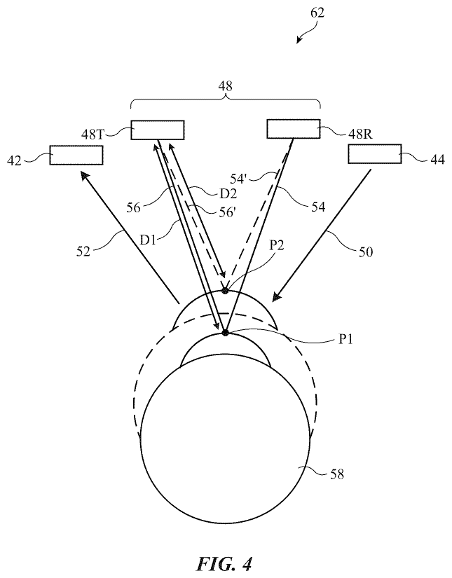

FIG. 4 is a top view of illustrative gaze tracking circuitry 62. As shown in FIG. 4, gaze tracking circuitry 62 may include one or more cameras such as camera 42, one or more light sources such as light source 44, and one or more range finders such as range finder 48. Range finder 48 may be configured to measure the distance to eye 58 (e.g., the distance to the point of specular reflection on the cornea, sometimes referred to as eye distance), sometimes referred to as eye distance). As the user's gaze moves around (e.g., from position P1 to position P2), the distance to eye 58 may change. At position P1, range finder 48 may measure a distance D1 to eye 58. At position P2, range finder 48 may measure a distance D2 to eye 58.

Range finder 48 may include one or more transmitters such as transmitter 48T and one or more receivers such as receiver 48R. Transmitter 48T may be configured to emit signal 56 toward the user's eye 58. Signal 56 may reflect off of eye 58 and reflected signal 54 may be detected by receiver 48R. If desired, range finder 48 may include more than one transmitter 48T and/or more than one receiver 48R. For example, range finder 48 may include a second transmitter 48T and a second receiver 48R for redundancy. Arrangements in which range finder 48 includes three or more transmitters 48T and/or three or more receivers 48R may also be used.

In some arrangements, one device may serve as both transmitter 48T and receiver 48R. For example, a flexible membrane in a transducer may be used to detect ultrasonic signals (when serving as receiver 48R) and may also be used to emit ultrasonic signals (when serving as transmitter 48T). As another example, a self-mixing interferometer may also serve as both transmitter 48T and receiver 48R.

Range finder 48 may be any suitable sensor configured to measure distance. In arrangements where range finder 48 is an optical sensor (e.g., an optical sensor that gathers time-of-flight measurements, a self-mixing sensor, a light detection and ranging (lidar) sensor, a structured light sensor, a phased-based optical coherence tomography sensor, and/or a depth sensor based on a stereo imaging device that captures three-dimensional images, etc.), emitted signal 56 and reflected signal 54 may be optical signals. When range finder 48 is formed from a phased-based sensor such as an optical sensor based on optical coherence tomography, range finder 48 may be configured to achieve smaller resolvable time intervals than time-of-flight based sensors. In arrangements where range finder 48 is an ultrasonic sensor (e.g., one or more capacitive micromachined ultrasonic transducers, piezoelectric micromachined transducers, and/or other suitable ultrasonic transducers for emitting and/or detecting acoustic signals), emitted signal 56 and reflected signal 54 may be ultrasonic signals.

If desired, transmitter 48T may be co-located with receiver 48R. In other arrangements, transmitter 48T and receiver 48R may be mounted in different locations. Camera 42 and light source 44 may be co-located with one another or may be mounted in different locations. One or both of transmitter 48T and receiver 48R may be co-located with camera 42 and/or light source 44, or transmitter 48T and receiver 48R may be mounted separately from camera 42 and/or light source 44.

During operation, light source 44 may be used to emit light 50 toward eye 58. Light 50 may reflect off of eye 58 and reflected light 52 may be detected by camera 42. Emitted light 50 may create a glint on eye 58. Camera 42 may capture images of eye 58 including the glint created by light 50. Based on the captured images, gaze tracking circuitry 62 may determine the location of the glint and the location of the user's pupil. In some arrangements, there may be multiple light sources 44 that produce multiple glints on the user's eye. If there are a sufficient number of glints produced on eye 58, gaze control circuitry 62 can determine the shape and/or the location of the user's eye or a part of the user's eye (e.g., the user's cornea), which in turn can be used to determine gaze direction (e.g., without requiring range finder 48).

In some arrangements, such as when fewer light sources 44 are desired, or when a sufficient number of glints cannot be captured due to the shape of a particular user's eye, gaze control circuitry 62 may combine glint detection with distance sensing to determine gaze direction. In this type of scenario, a single light source 44 may produce a single glint on eye 58, and camera 42 may capture images of eye 58 including the single glint. Based on the captured images, gaze tracking control circuitry 62 may determine a position of the glint and a position of the pupil. Because the eye is mostly spherical (e.g., to first order), the glint on eye 58 will remain mostly in the same place as the eye moves around, but the position of the pupil relative to the glint will change as the gaze direction changes. In particular, as the eyeball moves around to different gaze directions (e.g., from position to P1 to position P2), the position of the pupil relative to the glint will change by a scaling factor that depends on the distance to the eyeball. By using range finder 48 to determine a distance to the eyeball at positions P1 and P2, gaze tracking circuitry 62 can determine this scaling factor and can therefore map the pupil and glint positions at P1 to a first gaze direction (e.g., based on distance D1) and the pupil and glint positions at P2 to a second gaze direction (e.g., based on distance D2). In scenarios where no glints are visible to camera 42, multiple range finders 48 or a single range finder 48 can be combined with image information from camera 42 (e.g., eye features and/or facial features) which together enable an accurate assessment of the location and orientation of eye 58.

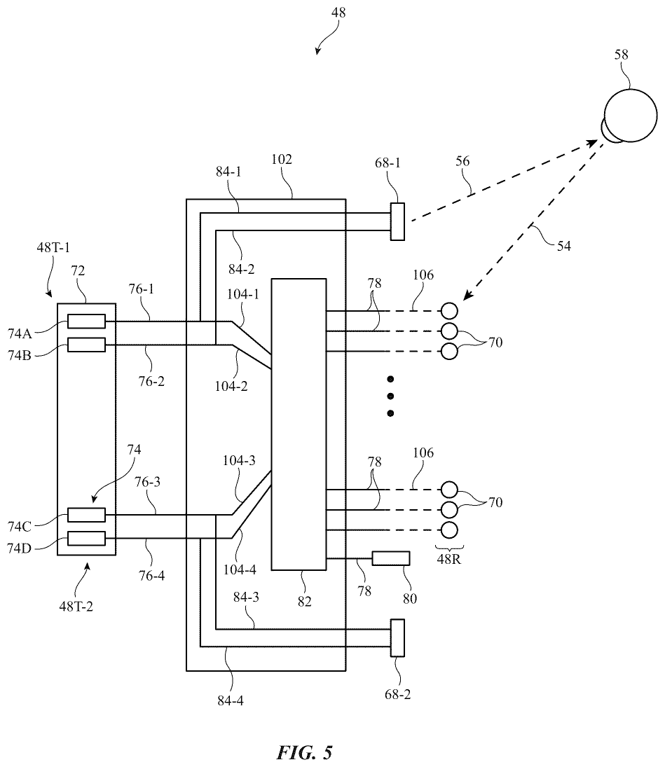

FIG. 5 is a schematic diagram of an illustrative range finder 48 that uses phase-based optical coherence tomography to determine the distance to a user's eye (e.g., as discussed in connection with FIG. 4). As shown in FIG. 5, range finder 48 may include one or more light sources such as transmitter 48T. Transmitter 48T may include one or more coherent light sources such as coherent light sources 74 on an integrated circuit such as integrated circuit die 72. Coherent light sources 74 may include one or more lasers such as lasers 74A, 74B, 74C, and 74D. Lasers 74 may be vertical cavity surface emitting lasers, distributed feedback (DFB) lasers, distributed Bragg reflector (DBR) lasers, microelectromechanical systems (MEMs) tunable lasers, thermally tuned lasers, or any other suitable coherent light source. Lasers 74 may be wavelength-tunable lasers configured to emit light while sweeping the wavelength of the light across a given range of tunability. For example, lasers 74 may be configured to emit light centered at 905 nm with 1.8 nm of tunability (e.g., from 904.1 nm to 905.9 nm). This example is merely illustrative. If desired, light emitted from lasers 74 may be centered around other wavelengths and/or may have other ranges of tunability.

Each laser 74 may emit a laser chirp with a given frequency sweep into a respective input of a beam splitter such as beam splitter 102. Beam splitter 102 may be a planar lightwave circuit (e.g., one or more passive optical components) or other suitable beam splitter for splitting light from lasers 72 into signal light such as signal light 56 and reference light such as reference light 106. Laser 72A may emit light into input 76-1 of beam splitter 102, laser 72B may emit light into input 76-2 of beam splitter 102, laser 74C may emit light into input 76-3 of beam splitter 102, and laser 74D may emit light into input 76-4 of beam splitter 102.

Beam splitter 102 may be configured to split light from lasers 74 into a signal arm headed toward eye 58 and a local oscillator arm that will interfere with the light collected from the signal arm. As shown in FIG. 5, beam splitter 102 may direct half of light from laser 74A to path 84-1 and the other half to path 104-1; may direct half of light from laser 74B to path 84-2 and the other half to path 104-2; may direct half of light from laser 74C to path 84-3 and the other half to path 104-3; and may direct half of light from laser 84D to path 84-4 and the other half to path 104-4. Light that is directed to paths 104-1, 104-2, 104-3, and 104-4 may serve as reference light (e.g., in the local oscillator arm that forms the fixed reference path), whereas light that is directed to paths 84-1, 84-2, 84-3, and 84-4 may serve as signal light (e.g., in the signal arm that forms the free space path to eye 58).

Paths 104-1, 104-2, 104-3, and 104-4 may serve as inputs to an optical coupler such as star coupler 82 for coupling reference light 106 from respective lasers 74 to one or more sensors such as image sensors 70. Star coupler 82 may have one or more outputs such as outputs 78. Star coupler 82 may have more outputs than inputs, if desired. For example, star coupler 82 may have four inputs and twelve outputs, may have eight inputs and twelve outputs, may have two inputs and ten outputs, and/or may have any other suitable number of inputs and outputs. Star coupler 82 may be configured to couple reference light 106 from inputs 104-1, 104-2, 104-3, and 104-4 to outputs 78. Outputs 78 may direct reference light 106 toward one or more image sensors 70. There may be one, two, three, ten, twelve, more than twelve, or less than twelve image sensors 70 in range finder 48. Each image sensor 70 may have a two-dimensional array of pixels. If desired, star coupler 82 may include an additional output 78 coupled to wavelength reference circuit 80 (e.g., for measuring the actual wavelength of each laser chirp).

Paths 84-1, 84-2, 84-3, and 84-4 may be configured to couple signal light 56 to a given emission aperture such as emission apertures 68-1 and 68-2 (sometimes referred to as exit apertures). If desired, range finder 48 may have more than two emission apertures (e.g., three, four, or more than four emission apertures). Emission apertures 68-1 and 68-2 may be positioned in different locations with respect to eye 58. Signal light 56 from paths 84-1 and 84-2 is coupled out of aperture 68-1 toward eye 58. Signal light 56 from paths 84-3 and 84-4 is coupled out of aperture 68-2 toward eye 58. Signal light 56 creates a specular reflection (e.g., a glint) on the user's eye and reflected signal light 54 is reflected toward one or more sensors 70 where it combines with reference light 106.

Each image sensor 70 may capture an image of the glint that is produced on eye 58 by signal light 56 from a given laser 74. If the free space path length to eye 58 is equal to the reference path length, then there will be no change to the image of the glint captured by image sensor 70. If the two path lengths are different, then constructive interference will occur at some frequencies of light and destructive interference will occur at other frequencies of light. The frequency at which the signal modulates between constructive and destructive interference is proportional to the difference in path length between the free space path that reflects off of eye 58 and the fixed reference path that has a known path length. In this way, the distance to eye 58 (e.g., distance D1 or distance D2 of FIG. 4) may be determined based on the interference pattern (e.g., the pattern of fringes) that is produced on image sensors 70.

In the example of FIG. 5, range finder 48 includes first and second emitters 48T that each include first and second lasers. For example, a first emitter 48T-1 having exit aperture 68-1 may include lasers 74A and laser 74B, and a second emitter 48T-2 having exit aperture 68-2 may include laser 74C and laser 74D. The use of multiple emitters 48T may allow for a diversity of path length measurements. If desired, there may be three, four, or more than four emitters 48T. Some of emitters 48T may be used for direct path measurements in which emitter 48T emits light on a free space path directly toward sensor 70 (e.g., without reflecting off of eye 58) in order to monitor for changes in the shape of glasses 10, which may be subject to daily wear and tear. In an arrangement with four emitters 48T, for example, range finder 48 may be configured to determine the x, y, and z location coordinates of each emitter 48T using direct path measurements.

The example of FIG. 5 in which each emitter 48T includes two lasers is merely illustrative. If desired, each emitter 48T may include three, four, or more than four lasers, or may include only one laser. The use of multiple lasers 74 in each emitter 48T allows for multiple laser chirps to be emitted in sequence during a given measurement, while allowing lasers 74 to have a rest period such as 1 ms of rest period after firing (e.g., for thermal management). Emitting multiple laser chirps in a given measurement may allow a first of the chirps to be used for determining glint location and subsequent chirps to be used for determining eye distance and its first one or two temporal derivatives (eye velocity and eye acceleration).

For example, during a given measurement, laser 74A may fire a first chirp having a first frequency sweep (e.g., a sweep from 905.9 nm to 904.1 nm) across a first sweep duration (e.g., 2 ms or other suitable duration). Beam splitter 102 may split the first chirp from laser 74A into signal light 56 which exits aperture 68-1 and reference light 106 which exits outputs 78 toward sensors 70. Image sensors 70 may capture one or more images of reflected signal light 54 and reference light 106. Based on the captured images, range finder 48 may determine the location of the glint (e.g., may determine which group of pixels on image sensor 70 includes the glint). This allows range finder 48 to only read out the particular group of pixels where the glint is located during subsequent chirps. For example, if sensor 70 includes an array of 50 pixels by 50 pixels, a subset region of 2 pixels by 4 pixels (corresponding to the glint location) may be read out in subsequent chirps for measuring path length and its derivatives (as an example).

After the first chirp, laser 74B may fire a second chirp having a second frequency sweep (e.g., from 904.1 nm to 905.9 nm) across a second sweep duration different from the first sweep duration (e.g., 0.5 ms or other suitable duration). Beam splitter 102 may split the first chirp from laser 74B into signal light 56 which exits aperture 68-1 and reference light 106 which exits outputs 78 toward sensors 70. Image sensors 70 may capture one or more images of reflected signal light 54 and reference light 106. If desired, only the subset of pixels where the glint is located (e.g., the subset of pixels identified from the first chirp) may be read out to measure the second chirp. Based on the captured images, range finder 48 may determine the free space path length and the corresponding distance to eye 58.

After the second chirp, laser 74A may again fire a third chirp having a third frequency sweep (e.g., from 905.9 nm to 904.1 nm) across a third sweep duration different from the first sweep duration (e.g., 1.5 ms or other suitable duration). Beam splitter 102 may split the third chirp from laser 74A into signal light 56 which exits aperture 68-1 and reference light 106 which exits outputs 78 toward sensors 70. Image sensors 70 may capture one or more images of reflected signal light 54 and reference light 106. If desired, only the subset of pixels where the glint is located (e.g., the subset of pixels identified from the first chirp) may be read out to measure the third chirp. Based on the captured images, range finder 48 may determine the first temporal derivative of the free space path length (e.g., eye velocity).

After the third chirp, laser 74B may again fire a fourth chirp having a fourth frequency sweep (e.g., from 904.1 nm to 905.9 nm) across a fourth sweep duration different from the first sweep duration (e.g., 0.5 ms or other suitable duration). Beam splitter 102 may split the fourth chirp from laser 74B into signal light 56 which exits aperture 68-1 and reference light 106 which exits outputs 78 toward sensors 70. Image sensors 70 may capture one or more images of reflected signal light 54 and reference light 106. If desired, only the subset of pixels where the glint is located (e.g., the subset of pixels identified from the first chirp) may be read out to measure the fourth chirp. Based on the captured images, range finder 48 may determine the second temporal derivative of the free space path length (e.g., eye acceleration).

In arrangements where range finder 48 includes multiple emitters 48T, emitters 48T may be active in alternate measurements. For example, lasers 74A and 74B of emitter 48T-1 may fire four chirps as described above in a first measurement, while lasers 74C and 74D of emitter 48T-2 are not active (e.g., not emitting light). In a second subsequent measurement, lasers 74C and 74D of emitter 48T-2 may fire four chirps (as described above for lasers 74A and 74B) while lasers 74A and 74B of emitter 48T-1 are not emitting light.

If desired, range finder 48 may use a signal processing technique such as PROFIT to process signals from sensor 70. With this type of technique, range finder 48 may determine the frequency of an isolated sinusoidal signal by taking N+1 data samples, then performing fast Fourier transforms on the first N samples and the last N samples. Each will show a peak around the location of the sinusoidal signal, but with a different phase. The frequency of the signal may be estimated based on the difference in phase alone (e.g., rather than using amplitude in bins outside the peak). This is merely illustrative, however. If desired, other signal processing techniques may be used to process signals from image sensor 70.

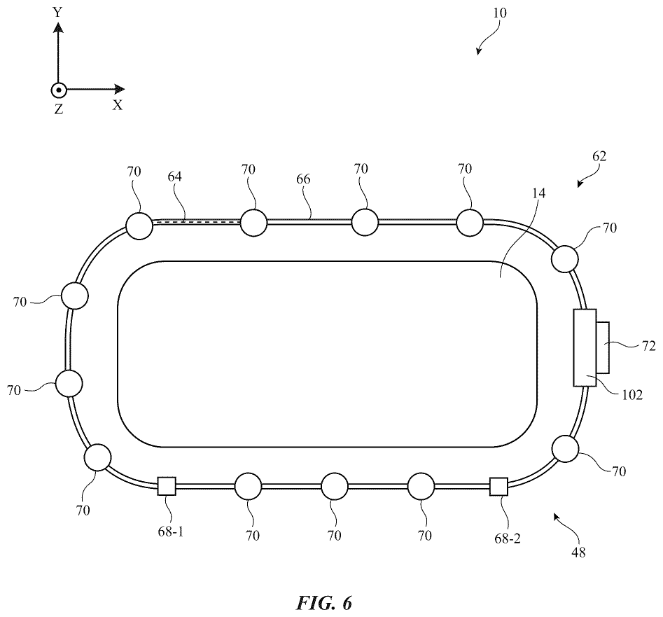

FIG. 6 is a rear view of device 10 showing illustrative locations for some of the components of FIG. 5. Image sensors 70 and emission apertures 68-1 and 68-2 may face the eye box and may be mounted to a support structure such as support structure 66. Support structure 66 may be a ring-shaped frame member that extends around the periphery of a user's eye. Support structure 66 may be part of housing 12 of device 10, part of optical module 40 of device 10, and/or may be a dedicated support structure for gaze tracking circuitry 62. In arrangements where device 10 includes a display such as display 14, support structure 66 may extend around some or all of the periphery of display 14, or support structure 66 may be mounted elsewhere in device 10 (e.g., surrounding lens 30 of FIG. 1, surrounding lens 50 of FIG. 1, etc.). Support structure 66 may have apertures for allowing emitted signal light 56 from emission apertures 68-1 and 68-2 to exit support structure 66 and may include apertures for allowing reflected signal light 54 to be detected by image sensors 70.

Range finder 48 may include one or more optical fibers such as optical fibers 64 in support structure 66. Optical fibers 64 may be configured to guide laser light (e.g., via total internal reflection) from beam splitter 102 to the desired location within support structure 66. For example, a first set of optical fibers 64 may be configured to guide reference light 106 from beam splitter 102 (e.g., from a given output 78 of star coupler 82) to respective image sensors 70. A second set of optical fibers 64 may be configured to guide signal light 56 from beam splitter 102 (e.g., from a respective one of paths 84-1, 84-2, 84-3, and 84-4) to respective emission apertures such as exit apertures 68-1 and 68-2. Reflected signal light 54 may interfere with reference light 106 and the corresponding interference pattern captured by image sensors 70 can be analyzed to determine glint location, eye speed, eye velocity, and/or eye acceleration as discussed in connection with FIG. 5.

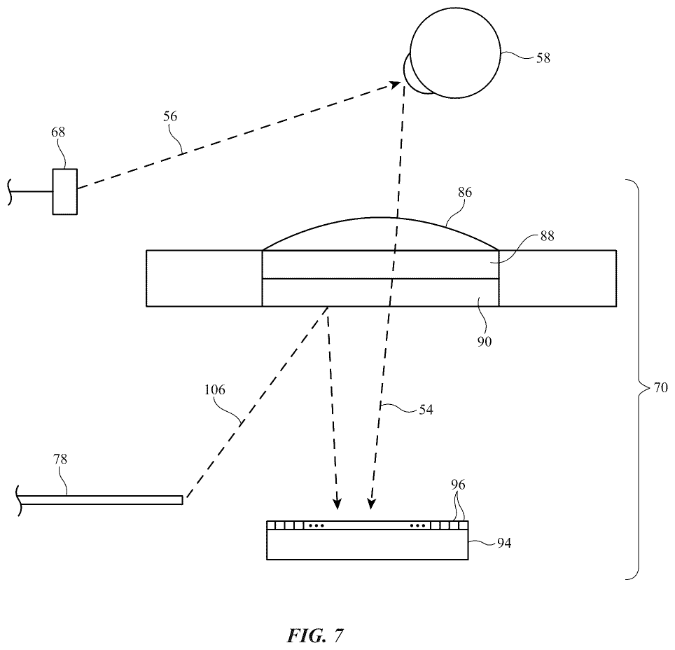

FIG. 7 is a side view of an illustrative image sensor 70 that may be used in range finder 48. As shown in FIG. 7, image sensor 70 may include a two-dimensional array of pixels 96 on a substrate such as semiconductor substrate 94. Image sensor 70 may be a complementary metal-oxide-semiconductor (CMOS) image sensor or other suitable image sensor. If desired, image sensor 70 may include one or more additional optical components overlapping pixels 96 such as lens 86, phase grating 88, and holographic optical element 90. Lens 86 may be used to focus reflected signal light 54 onto pixels 96. Phase grating 88 (sometimes referred to as diffraction grating 88) may be interposed between holographic optical element 90 and lens 86.

Holographic optical element 90 may be configured to couple reference light out of output 78 at an angle that is mostly parallel to incoming reflected signal light 54. Holographic optical element 90 may be a relatively sparse hologram with a small duty cycle (e.g., a metal pattern with 5% density or other suitable density). Making the reference wavefront parallel or mostly parallel to the signal wavefront reduces the spatial frequency of fringes between the two light paths and helps ensure that the interference pattern can be captured by pixels 96 even when pixels 96 are not overly small (e.g., 3 microns by 3 microns or other suitable size). Permitting relatively large pixels can help reduce the number of pixels that need to be digitized, thus saving power.

Phase grating 88 may be configured to split the specular reflection (e.g., the glint) on eye 58 into two spots on image pixels 96. Depending on whether the free space path is longer or shorter than the fixed reference path, one of the spots will lead the other (e.g., by 160 degrees of phase). Range finder 48 can therefore disambiguate the sign (e.g., positive or negative) associated with the difference in path length between the free space path and the fixed reference path. For example, if range finder 48 measures a one millimeter difference in path length, phase grating 88 may be used to determine if the free space path is one millimeter shorter or one millimeter longer than the fixed reference path based on which spot leads the other on pixels 96.

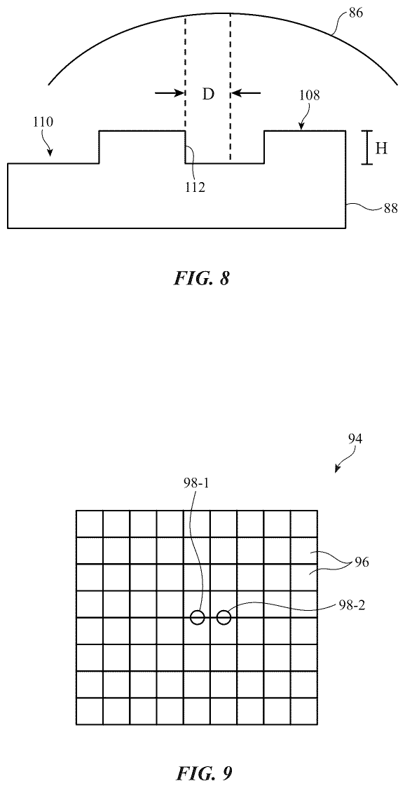

FIG. 8 is a side view of phase grating 88 and lens 86 of range finder 48. Phase grating 88 may be a surface relief grating formed from modulations in the thickness of a medium (e.g., having ridges 108 and troughs 110 in the medium that form fringes). If desired, phase grating 88 may be formed from two different types of plastic having a known etch depth and known refractive indices. The height H of ridges 108 relative to troughs 110 may be equal to about one half of the wavelength of the signal light so that one of the spots produced on pixels 96 leads the other.

As shown in FIG. 8, lens 86 may be laterally offset from the edges of phase grating 88. In particular, the vertex of lens 86 is laterally offset from all edges of phase grating 88 such as edge 112 of ridge 108. This lateral offset between lens 86 and phase grating 88 can be tuned to create asymmetry where one of spots on pixels 96 leads the other, as shown in FIG. 9. As shown in FIG. 9, phase grating 88 of FIG. 8 may split reflected signal light 54 into a double image, such as glint spot 98-1 and glint spot 98-2. Depending on whether the free space path is longer or shorter than the fixed reference path, one of glint spots 98-1 and 98-2 will lead the other. For example, glint spot 98-1 may lead glint spot 98-2 when the free space path length is longer than the fixed reference path length, whereas glint spot 98-2 may lead glint spot 98-1 when the free space path length is shorter than the fixed reference path length. Range finder 48 may therefore disambiguate the sign (e.g., positive or negative) associated with the calculated path length difference based on which of glint spots 98-1 and 98-2 leads the other.

As described above, one aspect of the present technology is the gathering and use of information such as information from input-output devices. The present disclosure contemplates that in some instances, data may be gathered that includes personal information data that uniquely identifies or can be used to contact or locate a specific person. Such personal information data can include demographic data, location-based data, telephone numbers, email addresses, social media information, home addresses, data or records relating to a user's health or level of fitness (e.g., vital signs measurements, medication information, exercise information), date of birth, username, password, biometric information, or any other identifying or personal information.

The present disclosure recognizes that the use of such personal information, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States, collection of or access to certain health data may be governed by federal and/or state laws, such as the Health Insurance Portability and Accountability Act (HIPAA), whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to provide certain types of user data. In yet another example, users can select to limit the length of time user-specific data is maintained. In addition to providing “opt in” and “opt out” options, the present disclosure contemplates providing notifications relating to the access or use of personal information. For instance, a user may be notified upon downloading an application (“app”) that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of information that may include personal information data to implement one or more various disclosed embodiments, the present disclosure also contemplates that the various embodiments can also be implemented without the need for accessing personal information data. That is, the various embodiments of the present technology are not rendered inoperable due to the lack of all or a portion of such personal information data.

Physical environment: A physical environment refers to a physical world that people can sense and/or interact with without aid of electronic systems. Physical environments, such as a physical park, include physical articles, such as physical trees, physical buildings, and physical people. People can directly sense and/or interact with the physical environment, such as through sight, touch, hearing, taste, and smell.

Computer-generated reality: in contrast, a computer-generated reality (CGR) environment refers to a wholly or partially simulated environment that people sense and/or interact with via an electronic system. In CGR, a subset of a person's physical motions, or representations thereof, are tracked, and, in response, one or more characteristics of one or more virtual objects simulated in the CGR environment are adjusted in a manner that comports with at least one law of physics. For example, a CGR system may detect a person's head turning and, in response, adjust graphical content and an acoustic field presented to the person in a manner similar to how such views and sounds would change in a physical environment. In some situations (e.g., for accessibility reasons), adjustments to characteristic(s) of virtual object(s) in a CGR environment may be made in response to representations of physical motions (e.g., vocal commands). A person may sense and/or interact with a CGR object using any one of their senses, including sight, sound, touch, taste, and smell. For example, a person may sense and/or interact with audio objects that create 3D or spatial audio environment that provides the perception of point audio sources in 3D space. In another example, audio objects may enable audio transparency, which selectively incorporates ambient sounds from the physical environment with or without computer-generated audio. In some CGR environments, a person may sense and/or interact only with audio objects. Examples of CGR include virtual reality and mixed reality.

Virtual reality: A virtual reality (VR) environment refers to a simulated environment that is designed to be based entirely on computer-generated sensory inputs for one or more senses. A VR environment comprises a plurality of virtual objects with which a person may sense and/or interact. For example, computer-generated imagery of trees, buildings, and avatars representing people are examples of virtual objects. A person may sense and/or interact with virtual objects in the VR environment through a simulation of the person's presence within the computer-generated environment, and/or through a simulation of a subset of the person's physical movements within the computer-generated environment.

Mixed reality: In contrast to a VR environment, which is designed to be based entirely on computer-generated sensory inputs, a mixed reality (MR) environment refers to a simulated environment that is designed to incorporate sensory inputs from the physical environment, or a representation thereof, in addition to including computer-generated sensory inputs (e.g., virtual objects). On a virtuality continuum, a mixed reality environment is anywhere between, but not including, a wholly physical environment at one end and virtual reality environment at the other end. In some MR environments, computer-generated sensory inputs may respond to changes in sensory inputs from the physical environment. Also, some electronic systems for presenting an MR environment may track location and/or orientation with respect to the physical environment to enable virtual objects to interact with real objects (that is, physical articles from the physical environment or representations thereof). For example, a system may account for movements so that a virtual tree appears stationery with respect to the physical ground. Examples of mixed realities include augmented reality and augmented virtuality. Augmented reality: an augmented reality (AR) environment refers to a simulated environment in which one or more virtual objects are superimposed over a physical environment, or a representation thereof. For example, an electronic system for presenting an AR environment may have a transparent or translucent display through which a person may directly view the physical environment. The system may be configured to present virtual objects on the transparent or translucent display, so that a person, using the system, perceives the virtual objects superimposed over the physical environment. Alternatively, a system may have an opaque display and one or more imaging sensors that capture images or video of the physical environment, which are representations of the physical environment. The system composites the images or video with virtual objects, and presents the composition on the opaque display. A person, using the system, indirectly views the physical environment by way of the images or video of the physical environment, and perceives the virtual objects superimposed over the physical environment. As used herein, a video of the physical environment shown on an opaque display is called “pass-through video,” meaning a system uses one or more image sensor(s) to capture images of the physical environment, and uses those images in presenting the AR environment on the opaque display. Further alternatively, a system may have a projection system that projects virtual objects into the physical environment, for example, as a hologram or on a physical surface, so that a person, using the system, perceives the virtual objects superimposed over the physical environment. An augmented reality environment also refers to a simulated environment in which a representation of a physical environment is transformed by computer-generated sensory information. For example, in providing pass-through video, a system may transform one or more sensor images to impose a select perspective (e.g., viewpoint) different than the perspective captured by the imaging sensors. As another example, a representation of a physical environment may be transformed by graphically modifying (e.g., enlarging) portions thereof, such that the modified portion may be representative but not photorealistic versions of the originally captured images. As a further example, a representation of a physical environment may be transformed by graphically eliminating or obfuscating portions thereof. Augmented virtuality: an augmented virtuality (AV) environment refers to a simulated environment in which a virtual or computer generated environment incorporates one or more sensory inputs from the physical environment. The sensory inputs may be representations of one or more characteristics of the physical environment. For example, an AV park may have virtual trees and virtual buildings, but people with faces photorealistically reproduced from images taken of physical people. As another example, a virtual object may adopt a shape or color of a physical article imaged by one or more imaging sensors. As a further example, a virtual object may adopt shadows consistent with the position of the sun in the physical environment.

Hardware: there are many different types of electronic systems that enable a person to sense and/or interact with various CGR environments. Examples include head mounted systems, projection-based systems, heads-up displays (HUDs), vehicle windshields having integrated display capability, windows having integrated display capability, displays formed as lenses designed to be placed on a person's eyes (e.g., similar to contact lenses), headphones/earphones, speaker arrays, input systems (e.g., wearable or handheld controllers with or without haptic feedback), smartphones, tablets, and desktop/laptop computers. A head mounted system may have one or more speaker(s) and an integrated opaque display. Alternatively, a head mounted system may be configured to accept an external opaque display (e.g., a smartphone). The head mounted system may incorporate one or more imaging sensors to capture images or video of the physical environment, and/or one or more microphones to capture audio of the physical environment. Rather than an opaque display, a head mounted system may have a transparent or translucent display. The transparent or translucent display may have a medium through which light representative of images is directed to a person's eyes. The display may utilize digital light projection, OLEDs, LEDs, uLEDs, liquid crystal on silicon, laser scanning light sources, or any combination of these technologies. The medium may be an optical waveguide, a hologram medium, an optical combiner, an optical reflector, or any combination thereof. In one embodiment, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

The foregoing is merely illustrative and various modifications can be made to the described embodiments. The foregoing embodiments may be implemented individually or in any combination.

What is claimed is:

1. Eyewear, comprising:
a support structure;
first and second lenses mounted to the support structure; and
gaze tracking circuitry configured to track gaze direction, wherein the gaze tracking circuitry comprises:
a coherent light source configured to emit infrared light;
a beam splitter configured to split the infrared light into signal light that travels a free space path and reference light that travels a fixed reference path, wherein the signal light creates an eye glint; and
an image sensor configured to capture images of the reference light and the signal light including the eye glint, wherein eye distance is determined based on the captured images.

2. The eyewear defined in claim 1 wherein the coherent light source comprises a vertical cavity surface emitting laser and wherein the infrared light is swept across a range of wavelengths during emission.

3. The eyewear defined in claim 1 wherein the beam splitter comprises a planar lightwave circuit that splits the infrared light into the signal light and the reference light.

4. The eyewear defined in claim 1 wherein the beam splitter comprises a star coupler having an input that receives the reference light and an output that directs the reference light toward the image sensor.

5. The eyewear defined in claim 1 wherein the image sensor comprises a two-dimensional array of pixels.

6. The eyewear defined in claim 5 wherein the image sensor comprises a lens overlapping the two-dimensional array of pixels.

7. The eyewear defined in claim 6 wherein the image sensor comprises a phase grating interposed between the two-dimensional array of pixels and the lens and wherein a vertex of the lens is laterally offset from all edges of the phase grating.

8. The eyewear defined in claim 7 wherein the image sensor comprises a holographic optical element interposed between the phase grating and the array of pixels, wherein the holographic optical element is configured to redirect the reference light to be parallel to the signal light.

9. The eyewear defined in claim 1 wherein the coherent light source and the image sensor have different locations.

10. The eyewear defined in claim 1 wherein the image sensor is one of multiple image sensors that detect the signal light and the reference light from the coherent light source.

11. A distance sensor, comprising:
first and second emitters, wherein each emitter comprises a first laser that fires a first chirp and a second laser that fires a second chirp after the first chirp;
a beam splitter that splits the first and second chirps into signal light and reference light, wherein the signal light travels a free space path length and creates a specular reflection while the reference light travels a fixed reference path length; and
an image sensor configured to capture images of the signal light and the reference light of the first and second chirps, wherein a location of the specular reflection is determined based on the first chirp and wherein a difference between the free space path length and the fixed reference path length is determined based on the second chirp.

12. The distance sensor defined in claim 11 wherein the first and second emitters are configured to be active during alternate measurements.

13. The distance sensor defined in claim 11 wherein the image sensor comprises a pixel array and a lens overlapping the pixel array.

14. The distance sensor defined in claim 13 further comprising a phase grating interposed between the lens and the pixel array, wherein the phase grating is configured to create a double image of the specular reflection on the pixel array that indicates whether the free space path length is longer or shorter than the fixed reference path length.

15. The distance sensor defined in claim 13 further comprising a holographic optical element interposed between the lens and the pixel array, wherein the holographic optical element is configured to redirect the reference light to be parallel to the signal light.

16. A head-mounted device, comprising:
a display configured to present an image;
a lens through which the image is viewable from an eye box;
image sensors distributed around a periphery of the display; and
first and second emitters configured to emit coherent infrared light that is split into signal light headed toward the eye box and reference light headed toward the image sensors, wherein the signal light is specularly reflected at the eye box and wherein the specularly reflected signal light combines with the reference light and creates a corresponding interference pattern that is captured by the image sensors and used to determine eye distance.

17. The head-mounted device defined in claim 16 wherein each of the first and second emitters comprises at least first and second lasers, the head-mounted device further comprising first optical fibers that guide the reference light from the first and second lasers to the image sensors and second optical fibers that guide the signal light from the first and second lasers to respective exit apertures facing the eye box.

18. The head-mounted device defined in claim 16 wherein the image sensors each comprise an array of pixels, wherein the first laser is configured to fire a first chirp of the coherent infrared light and the second laser is configured to fire a second chirp of the coherent infrared light after the first chirp, wherein the first chirp is used to identify a portion of the array of pixels that includes a glint created by the specularly reflected signal light, and wherein the second chirp is used to determine the eye distance.

19. The head-mounted device defined in claim 18 wherein only the portion of the array of pixels that includes the glint is read out for processing the second chirp.

20. The head-mounted device defined in claim 16 wherein at least one of the image sensors comprises:
a pixel array;
a lens overlapping the pixel array;
a phase grating interposed between the pixel array and the lens; and
a holographic optical element interposed between the phase grating and the pixel array.
